(19) United States
(12) Patent Application Publication
(10) Pub. No.: US 2006/0110332 A1
(43) Pub. Date: May 25, 2006

(54) Agent for medicamentous treatment of acute and chronic pain

(75) Inventors: Gary Lewin, Berlin (DE); Jung-Bum Shin, Berlin (DE)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: Max-Delbruck-Centrum Fur Molekulare Medizin, Berlin (DE)

(21) Appl. No.: 10/518,594

(22) PCT Filed: Jun. 20, 2003
(86) PCT No.: PCT/EP03/06551

§ 371 Date: Nov. 8, 2005

(30) Foreign Application Priority Data
Jun. 19, 2002 (DE) 102 27 511.4

(51) Int. Cl. A61K 9/14; A61K 9/48; A61K 9/20; A61L 9/04

(52) U.S. Cl. 424/46; 514/355; 424/451; 424/464

(57) ABSTRACT

The invention relates to an agent for the medicamentous treatment of acute and chronic pain, in particular of allodynia and hyperalgesia. Fields of application of the invention are medice and the pharmaceutical industry. A new pharmaceutical composition for the treatment of acute and/or chronic pain, in particular allodynia and hyperalgesiais provided, the pharmaceutical composition comprising calcium channel blockers which are suitable for blocking voltage-dependent calcium channels, in particular of the T-type, more preferably the CaV3.2 channel and/or of the L-type. Mibefradil and dihydropyridines can, for instance, be used as calcium channel blockers.

The invention relates to an agent for medicamentous treatment of acute and chronic pain, in particular of allodynia and hyperalgesia.

This invention can be applied in the fields of medicine and pharmaceutical industry.

Mechanical stimuli of different subjective sensation are perceived through the skin, which is the largest sense organ in humans. The perception of sensation ranges from soft contact, pressure and tickle to perceptions of pain due to strong mechanical impact.

Pain conditions that are triggered by a stimulus that, under normal conditions, does not cause any pain, are called allodynic. Prominent examples thereof are a hypersensitivity of the skin due to a sunburn, an inflammation or a trauma. In order to distinguish it from hyperalgesia, it is important to note that allodynia is always connected with a change in the sensory modality. In other words, in the case of allodynia, it is, for instance, no longer possible to differentiate between the sensation modalities of a “soft touch” and “pain”, or, in other words, that a stimulus which is normally not painful causes pain. This means that there is a loss in the specificity of the sensory modalities. In contrast, in the case of hyperalgesia, the quality of the sensory sensation has not changed. A touch is still perceived as a touch and pain is still perceived as pain. One, however, is more sensitive and quantitatively feels a more intensive touch or pain. The transition from allodynia to hyperalgesia, however, is mostly gradual. It is at present assumed that both the peripheral and the central sensitisation of the pain system contribute to the two pathological conditions (Julius D. Basbaum A I. (2001) Molecular mechanisms of nociception. Nature 2001 413(6852):203-10; Textbook of Pain, (1999) ed. Wall P D and Melzack R. Philadelphia, Pa., W B Saunders, ISBN 0-443-06252-8).

Neuropathic pain, such as hyperalgesia and allodynia both occur as symptoms of many different and varied diseases and injuries (Epidemiology of Pain (1999), IASP Press, Editors: Ian K. Crombie, Peter R. Croft, Steven J. Linton, Linda LeResche, Michael von Korffm, ISBN 0-931092-25-6). Examples thereof include syndromes such as rheumatoid arthritis, cancer pain, sport injuries, chronic and acute back pain, herpes zoster and post-surgical pain requiring intensive treatment. The treatment of neuropathic pain is often very difficult because of the multiple underlying mechanisms that are poorly understood. Any novel analgetic target has a great therapeutic potential.

Allodynia is a pathological condition in which the person perceives soft mechanical stimuli on the skin as pain, which, under normal conditions, are merely perceived as a soft contact of tickling. This is probably due to a change in the connections in the spinal cord.

The different stimuli are registered by the endings of sensory neurons that are present in the spinal ganglion and the peripheral terminations of which extend to the ends of the extremities.

Due to the requirements as to the processing of different mechanical stimuli, this group of neurons has a very heterogeneous population. They differ from each other in terms of the conduction velocity of their axons, the cell size, the threshold for mechanical generation of stimulation and their adaptation behaviour:
1. A-β fibres (more than 10 m/s): slowly adapting SA fibres and rapidly-adapting RA fibres (both tactile receptors)
2. A-delta fibres (1-10 m/s): AM fibres (nociceptor) and D-hair mechanoreceptors (highly sensitive tactile receptors)
3. C-fibres (under 1 m/s) (nociceptors) Johnson (Kenneth O. Johnson, 2001, The roles and functions of cutaneous mechanoreceptors, Current opinion in neurobiology, 11; 455-461, for mouse: Koltzenburg M, Stucky C L, Lewin G R. Receptive properties of mouse sensory neurons innervating hairy skin. J Neurophysiol. 1997 October; 78(4):1841-50) summarises the cutaneous mechanoreceptors in humans.

Currently, two classes of pharmaceuticals are used for treating allodynia and hyperalgesia, namely the class of non-steroidal anti-inflammatory drugs (NSAID) (such as indomethacin and aspirin) and the opiates. The latter have an effect on the central nervous system and they can only be applied to a limited extent due to the known side-effects such as dependency and tolerance. The NSAIDs are effective in the periphery and are therefore safer and more effective in many cases.

In chronic conditions such as rheumatoid arthritis, however, NSAIDs have not turned out to be effective so that further targets for nociceptive treatment have to be found. In this way, on the one hand, it would be possible to develop agents that are more effective than the NSAIDs or, on the other hand, these classes of pharmaceuticals could be supplemented due to their different mode of activity and they could mutually potentate their analgesic effect.

Thus, the technical problem underlying the invention is to develop a new agent for the medicamentous treatment of acute and chronic pain, in particular of allodynia and hyperalgesia.

This technical problem is solved according to the claims.

According to the present invention, it was surprisingly found that voltage-dependent calcium channels are involved in the transduction of mechanical stimuli by nociceptive/non-nociceptive neurons. As a consequence, a blockade of the mechanical sensitivity of the skin by means of calcium channel blockers such as mibefradil or a dihydropyridine derivative is the basis of the invention and thus offers a totally new treatment of pain such as allodynia and hyperalgesia.

A new pharmaceutical composition for the treatment of acute and/or chronic pain, in particular allodynia and hyperalgesia is provided for, comprising calcium channel blockers which are suitable for blocking voltage-dependent calcium channels, in particular of the T-type, most preferably the CaV3.2 channel, and/or of the L-type. Mibefradil and dihydopyridine are preferred calcium channel blockers to be used in accordance with the present invention.

Further calcium channel blockers including T-type channel blockers are known in the art. Such substances which can be used in accordance with the present invention include 1,4-dihydropyridine derivatives as disclosed in WO98/31680, EP0164588 and EP0158211, succinimide derivatives like methylphenylsuccinimide, diphenylmethylpiperazine derivatives like 7-[[4-[bis(4-fluorophenyl)-methyl]-1-piperazinyl]methyl]-2-[(2-hydroxyethyl) amino]4-(1-methylethyl)-2,4,6-cycloheptatrien-1-one (U92032; Pharmacia and Upjohn), flunarizine, efonidipine, pimozide, zonisamide, depacon, amiloride and/or valproic acid.

The agent of the invention can, amongst others, be used in the treatment of pain associated with rheumatoid arthritis, the formation and growth of tumours, injuries, back pain, herpes zoster and post-surgical pain.

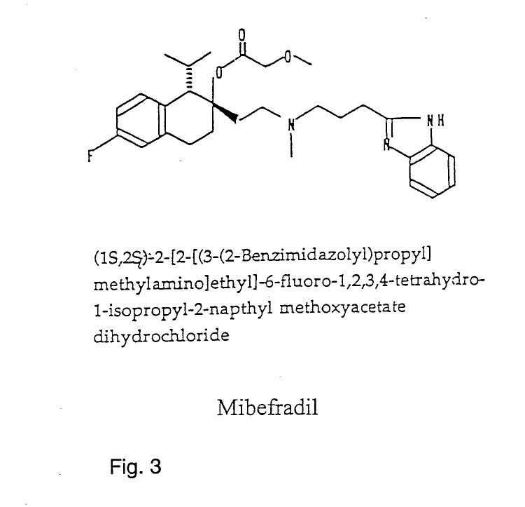

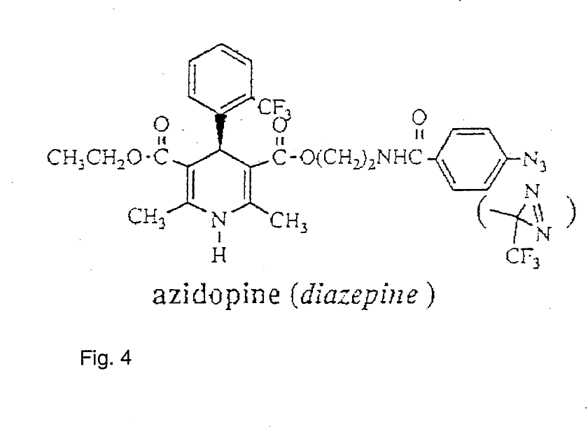

The agent can be applied in local, oral, parenteral, inhalative or intranasal form, in any pharmaceutically acceptable form. In accordance with a preferred embodiment of the invention, the calcium channel blocker is mibefradil (see FIG. 3), its pharmaceutically acceptable analogues, salts and esters or dihydropyridines, such as diazepin, as well as their pharmaceutically acceptable analogues (see FIG. 4). In another embodiment of the invention, for extending the possibilities of therapy, ointments, gels or cremes and solutions or suspensions are used as local forms of application. The pharmaceutical composition of the invention can furthermore be included into a tape or can be applied in form of a spray, in particular a nasal spray.

Another advantage of the invention is that it can be applied for the systemic treatment of pain. For extending the possibilities of therapy, tablets, capsules, coated tables, granulates, effervescent tablets, juice, syrup, suspensions or solutions can be used as oral forms of application. In this case, the drug form used is formed of biologically utilizable or biodegradable substances, wherein the biological materials are proteins or proteides, lipids or lipoids, carbohydrates or polysaccharides or mixtures of several of such materials.

For extending the possibilities of therapy, in addition, to the pharmaceutical composition of the invention, at least one other analgetic, preferably of the NSAID class can be used. In this way, it is possible to supplement the different biophases and to enhance the analgetic effect.

Preferably, the concentration of mibefradil is between 1 and 10 μM, more preferably 3 to 7 and most preferably 5 μM.

### EXAMPLES

The invention described is now explained in more detail by way of the following examples. The person skilled in the art can take various other embodiments from the present description. Attention, however, is drawn to the fact that the examples and the description are merely intended to explain and not to limit the invention.

Analysis of the occurrence and influence of voltage-dependent calcium channels in D-hair mechanoreceptors

With regard to the research of pain, amongst the various kinds of mechanoreceptors, in particular the so-called D-hair mechanoreceptors which are an excellent example of tactile receptors thanks to their high sensitivity. Stucky et al. found that in mice which lacked the gene for the neurotrophin NT-4 a loss of D-hair mechanoreceptors occurs (Stucky C L, DeChiara T, Lindsay R M, Yancopoulos G D, Koltzenburg M., Neutrotrophin 4 is required for the survival of a subclass of hair follicle receptors. J. Neurosci. 1998 Sep. 1; 18(17):7040-6). In the present invention, these mice were used for the detection of genes which are specifically expressed in D-hair and could therefore be important for their function. For this purpose, the gene expression of WT and NT-4 ko (knock-out) mice were analysed in a comparison to detect genes that were under-regulated in NT-4 mice. As, with regard to the skin sensory system, the only difference between WT and NT-4 ko mice is the loss of D-hair, these under-regulated genes were potential candidates for D-hair specific genes. A combination of gene chip analysis and DNA subtraction methods were used for expression studies. A combined analysis of the gene chip expression data and the DNA subtraction data resulted in 29 genes which are most probably under-regulated in NT-4 ko mice. For detecting genes which are specifically expressed in a subtype of the spinal neurons, their expression pattern in the spinal ganglion was analysed. For this purpose, in-situ hybridisations with Dig labelled cRNA were carried out. D-hair specific genes should have been expressed in medium-size neurons and under-regulated in NT-4 ko mice.

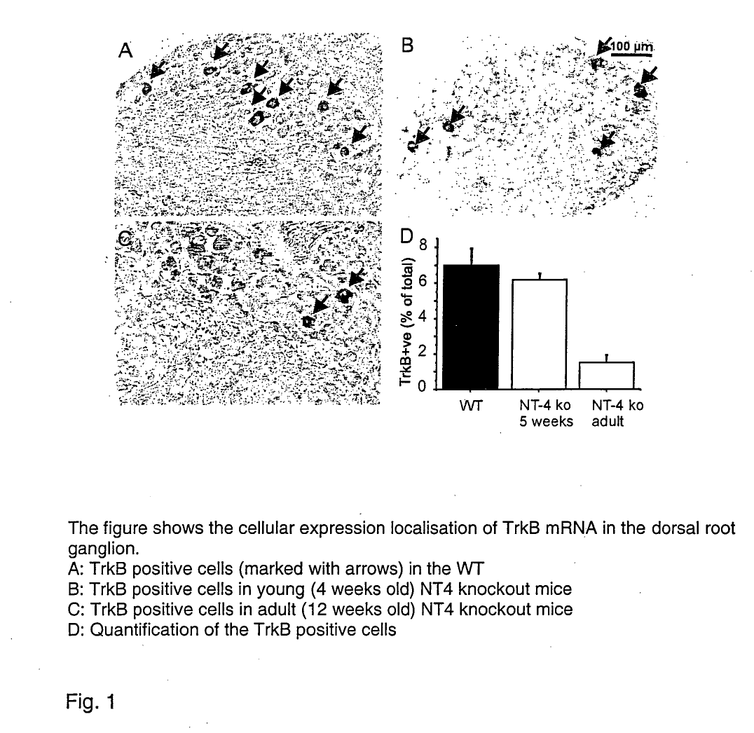

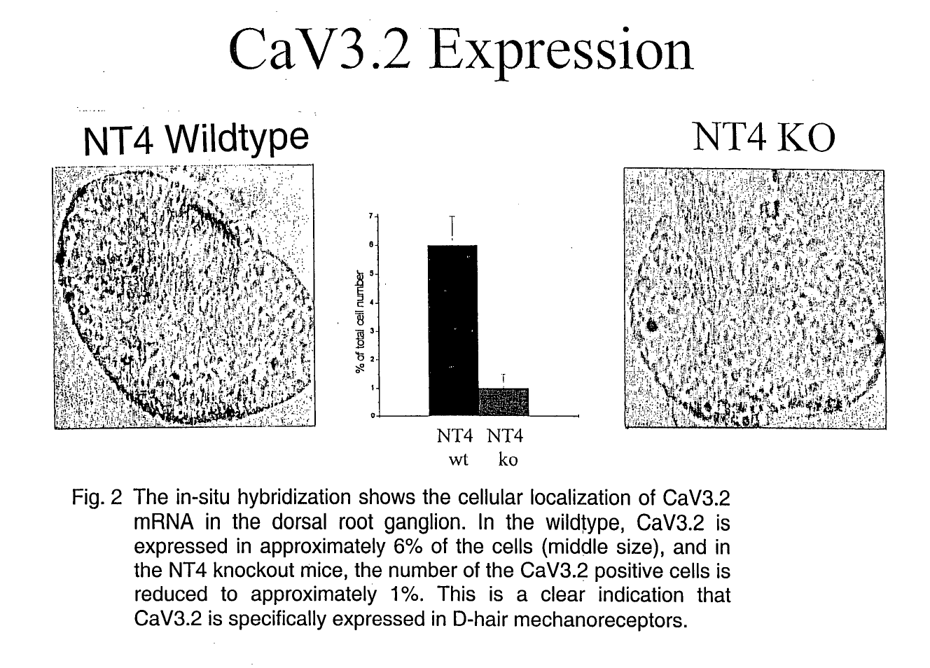

All genes were expressed in a neuronal manner. Two genes fulfilled the above-mentioned criteria for D-hair specificity. These were the genes TrkB and T-type calcium channel CaV3.2. (FIGS. 1 and 2)

TrkB ist the cellular receptor of NT-4 and BDNF and therefore an under-regulation of TrkB in NT-4 mice could be expected and confirmed the usefulness of the present experimental approach. The findings, however, that CaV3.2 is specifically expressed in D-hair mechanoreceptors is new and surprising as it has so far been unknown that calcium channels are involved in mechanosensation.

Although T-type calcium currents have already been identified in the eighties by means of electrophysiological studies with chick sensory neurons (Fox A P, Nowycky M C, Tsine R W, Kinetic and pharmacological properties distinguishing three types of calcium currents in chick sensory neurons. J. Physiol. 1987 December; 394:149-72), the genes were cloned only recently. The T-type subtype CaV3.2 consists of 2042 amino acids and was originally cloned in heart (therefore, its alternative name alpha1H) (Cribbs L L, Lee J H, Yang J, Satin J, Zhang Y, Daud A, Barclay J, Williamson M P, Fox M, Rees M, Perez-Reyes E. Cloning and characterization of alpha1H from human heart, a member of the T-type Ca2+ channel gene family. Circ. Res. 1998 Jul. 13; 83(1):103-9), but was later on also detected in kidney and liver, and, to a smaller extent, also in brain. The expression studies underlying the present invention showed that CaV3.2 is expressed very specifically in medium-size spinal ganglion cells. In the spinal ganglion, there are two kinds of medium-size neurons, the AM- and D-hair mechanoreceptors. Due to the findings obtained by the present invention, i.e. that, parallel to the loss of D-hair, also CaV3.2 positive neurons disappear in NT-4 ko mice, it is obvious that CaV3.2 is specifically expressed in D-hair.

The logical question was which role said calcium channel plays in D-hair mechanoreceptors and whether it is indispensable for their function. For these studies, two known calcium channel blockers, i.e. mibefradil and nickel, were used (Martin R L, Lee J H, Cribbs L L, Perez-Reyes E, Hanck D A, Mibefradil block of cloned T-type calcium channels. J. Pharmacol Exp Ther. 2000 October; 295(1):302-8 (Lee J H, Gomora J C, Cribbs L L, Perez-Reyes E. Nickel block of three cloned T-type calcium channels: low concentrations selectively block alpha1H. Biophys J. 1999 December; 77(6):3034-42). Mibefradil (see FIG. 3) is a non-dihydropyridine calcium channel antagonist that has a relatively high selectivity for T-type calcium channels. Until a few years ago, mibefradil was used for the alternative treatment of hypertension and angina pectoris (Frishman W H, Mibefradil: A New selective T-Channel Calcium Antagonist for Hypertension and Angina Pectoris. J Cardiovasc Pharmacol Ther. 1997 October; 2(4):321-330) (Brogden R N, Markham A.: Mibefradil. A review of its pharmacodynamic and pharmacokinetic properties, and therapeutic efficacy in the management of hypertension and angina pectoris. Drugs. 1997 November; 54(5):774-93. Review), but it was removed from the market due to its severe interaction with other medicaments (Clozel J P, Ertel E A, Ertel S I; Voltage-gated T-type Ca2+ channels and heart failure. Proc Assoc Am Physicians. 1999 September-October; 111(5):429-37. Review). The EC50 of mibefradil on cells is between 0.1 and 1 μM, depending on the cell system (Martin et al., 2000, see above). Mibefradil however has an almost identical effect on both T-type isotypes CaV3.1 und CaV3.3. The other blocker nickel has a 70 times higher EC50 on CaV3.2 than on the other isotypes (approx. 10 μM on CaV3.2 and 216 μM CaV3.3 and 25.0 μM on CaV3.3). Nickel, however, is cytotoxic so that it is only restrictedly suitable for pharmacologic analyses of living tissue and is even unsuitable for medicamentous use.

The use of the skin-nerve preparation, which the present invention is based on, allows for the electrophysiological analysis of the different neurofibre types which innervate the skin (Koltzenburg et al., 1997, see above). The saphenous nerve and the region of the skin that it innervates are prepared from freshly killed mice and are mounted in a bath of physiological buffer. After mechanical or electrical stimulation of the skin, the nerve signals can be received directly from the nerve. For determining the effective concentration, tests were carried out with different concentrations of mibefradil in the bath solution. The EC50 of mibefradil on cells is between 0.1 and 1 μM. At high concentrations of more than 25 μM mibefradil in the bath solution, there was an almost complete and unspecific blocking of the mechanical sensitivity. If the concentration was lowered to 3 μM, D-hair mechanoreceptors and, in part, also AM mechanoreceptors are specifically inhibited, the A-β fibres, however, not. The unspecific blockade at high concentrations may be due to the fact that mibefradil, at high concentrations, has a very unspecific effect on other ubiquitously expressed calcium channels. Such blocking is lost at lower concentrations. The blocking of some AM mechanoreceptors also at low concentrations of mibefradil could have two reasons. The first possibility is that AM mechanoreceptors, too, express CaV3.2. This, however, is not very likely based on the findings obtained by the in-situ hybridisation within the framework of the present invention. It may rather be assumed that a different isotype, i.e. CaV3.3 is expressed in AM mechanoreceptors. It was not possible to detect CaV3.3 in in-situ hybridisation experiments in spinal ganglia, but other groups reported the expression of this calcium channel in medium-size cells. And it has been known that the semi-effective concentration of mibefradil on CaV3.3 is almost identical to the one on CaV3.2. To sum up, it is concluded that the calcium channel CaV3.2 in spinal ganglia specifically expresses in D-hair mechanoreceptors and is therefore indispensable for its function. The discovery that the voltage-dependent calcium channels are involved in the transduction of mechanical stimuli by nociceptive and non-nociceptive neurons is new. Due to their physiological properties together with their specific localisation on the site of the mechanotransduction in the periphery, the calcium channels are ideal targets for pain therapy, which the invention is based on.

Functional Model

It is not very likely that this T-type calcium channel is the mechanosensitive ion channel that is responsible for the generation of the receptor potential. It is rather concluded that this calcium channel is a kind of signal enhancer. Moderately strong depolarisations which are triggered by soft mechanical stimuli do normally not surpass the threshold for the initiation of an action potential. It would be possible to achieve a signal enhancement by inserting an ion channel which is activated even at low voltages. This would be a simple explanation for the high sensitivity of D-hair mechanoreceptors. CaV3.2 has ideal prerequisites for fulfilling such a task as it is activated even at low voltages. Another feature of the D-hair receptors is their fast adaptation, i.e. they are only active at the beginning and at the end of a mechanical stimulus, i.e. they are acceleration receptors. A property of the CaV3.2 receptor is that it deactivates if these are stimuli in rapid succession, which is well compatible with the fast-adapting property of D-hair receptors.

Therapeutic Approach

The experiments of the invention were carried out on mice. Human CaV3.2 shows a very high genetic homology and is very likely to exert similar functions. The new findings that the use of a calcium channel blocker (e.g. with mibefradil even at low concentrations (25 μM or less)) virtually inhibits the entire cutaneous mechanotransduction, is of particular significance in the treatment of allodynia. These are pathological conditions in which the person perceives soft mechanical stimuli on the skin, which are normally merely perceived as soft touch or tickling, as pain. This is most probably due to a change in the neuronal connections in the spinal cord.

A blockade of the mechanical sensitivity of the skin by the systemic or topic addition of calcium channel blockers such as mibefradil is of great clinical significance in terms of the treatment of such conditions.

Extension of the Possibilities of Application

The data of the invention primarily relate to the calcium channel CaV3.2, a sub-type of the voltage-dependent calcium channels. It should, however, be noted that the blockade of other voltage-dependent calcium channels can also be used for the treatment of the cited pain. It has been found that the use of higher concentrations of mibefradil resulted in the complex blocking of other mechanoreceptors which have other calcium channels. The group of voltage-dependent calcium channels can be classified as follows:

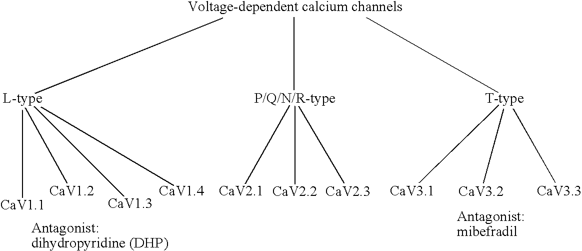

As a result, at higher concentrations, mibefradil blocks not only the D-hair mechanotransduction but also the entire mechanotransduction. This is probably due to a blocking of other voltage-dependent calcium channels, in particular the L- and N-type. N-type calcium channels are expressed in all sensory neurons, whereas the L-type is mainly expressed in small cells (Scroggs R S, Fox A P; Calcium current variation between acutely isolated adult rat dorsal root ganglion neurons of different size. J. Physiol. 1992 January; 445:639-58) and are blocked by mibefradil at higher concentrations (about 10-fold higher than necessary for T-type blocking) (Mehrke G, Zong X G, Flockerzi V, Hofmann F. The Ca(++)-channel blocker Ro 40-5967 blocks different T-type and L-type Ca++channels. J Pharmacol Exp Ther. 1994 December;271(3):1483-8). This is why also dihydropyridines, the more effective L-type blockers, can be used for the treatment of the mentioned conditions of pain. Dihydropyridines have already been used clinically, e.g. for the treatment of hypertension (Reuter H, Porzig H, Kokubun S, Prod'hom B.; Calcium channels in the heart. Properties and modulation by dihydropyridine enantiomers. Ann N Y Acad. Sci. 1988; 522:16-24. Review).

1. Pharmaceutical composition for the treatment of acute and/or chronic pain comprising calcium channel blockers that are capable of blocking voltage-dependent calcium channels.

2. Pharmaceutical composition as defined in claim 1 wherein the calcium channel is a T-type or L-type channel.

3. Pharmaceutical composition as defined in claim 1 for the treatment of allodynia or hyperalgesia.

4. Pharmaceutical composition according to claim 1 wherein the calcium channel blocker is mibefradil, its pharmaceutically acceptable analogues, salts or esters or a dihydropyridine.

5. Pharmaceutical composition according to claim 1 for the treatment of pain associated with rheumatoid arthritis, cancer, injuries, back pain, herpes zoster and post-operative pain.

6. Pharmaceutical composition according to claim 1 for the topical, oral, parenteral, inhalative or intranasal administration.

7. Pharmaceutical composition according to claim 6 in form of an ointment, gel, creme or a solution or suspension, or plaster.

8. Pharmaceutical composition according to claim 6 in form of a nasal spray or inhalator.

9. Method for the systemic therapy of pain, comprising administering a pharmaceutical composition comprising calcium channel blockers that are capable of blocking voltage-dependent calcium channels.

10. Pharmaceutical composition according to claim 1 wherein the composition is in a drug form for oral administration, wherein the form is selected from the group consisting of a tablet, a capsule, a coated tablet, a granulate, a juice, a syrup, a suspension, and a solution.

11. Pharmaceutical composition according to claim 1 wherein the drug form comprises biologically utilizable or biodegradable substances selected from the group consisting of proteins or proteides, lipids or lipoids, carbohydrates, polysaccharides and mixtures thereof.

12. Pharmaceutical composition according to claim 1 which further comprises one other pain killer.

13. Pharmaceutical composition according to claim 12 wherein the other pain killer is selected from the group consisting of an NSAID, a 5HT_{1D} agonist, a dopamin D₂ receptor antagonist, a secale alcaloid, a beta blocker, a calcium channel blocker, and a neurokinin antagonist.

14. Pharmaceutical composition according to claim 12 wherein the NSAID is ibuprofen, meoxicam, indomethacin or naporxen.

15. Pharmaceutical composition according to claim 12 wherein the 5HT_{1D} agonist is sumatriptan, MK-452, naratriptan or 311C.

16. Pharmaceutical composition according to claim 12 wherein the dopamin D₂ receptor antagonist is metoclopramid.

17. Pharmaceutical composition according to claim 12 wherein the secale alcaloid is ergotamin, dihydroergotamin or metergolin.

18. Pharmaceutical composition according to claim 12 wherein the beta blocker is propranolol or metoprolol.

19. Pharmaceutical composition according to claim 12 wherein the calcium channel blocker is flunarizin or lomerizin.

20. Pharmaceutical composition according to claim 12 wherein the other pain killer is selected from the group consisting of acetylsalicylic acid, paracetamol, clonidin, methysergid, dotarizin, lisurid, pizotifen, valproat, aminotraptilin CP-122,288, and UK 116,044.
